(19) United States
(12) Patent Application Publication
(10) Pub. No.: US 2021/0389483 A1
(43) Pub. Date: Dec. 16, 2021

(54) Fast Gamma-Ray Interaction-Position Estimation

(71) Applicant: Arizona Board of Regents on Behalf of the University of Arizona, Tucson, AZ (US)

(72) Inventors: Xin LI, Tucson, AZ (US); Lars FURENLID, Tucson, AZ (US)

(73) Assignee: Arizona Board of Regents on Behalf of the University of Arizona, Tucson, AZ (US)

(21) Appl. No.: 17/286,727

(22) PCT Filed: Oct. 18, 2019
(86) PCT No.: PCT/US19/57067

§ 371 Date: Apr. 19, 2021

Related U.S. Application Data
(60) Provisional application No. 62/748,165, filed on Oct. 19, 2018.

(51) Int. Cl. G01T 1/29; A61B 6/03; G01T 1/20

(52) U.S. Cl. CPC G01T 1/2985; G01T 1/2006; A61B 6/037

(57) ABSTRACT

Disclosed are fast gamma-ray interaction-position estimation methods using k-d tree searching. Compared with conventional methods, the methods disclosed herein achieve both high levels of speed and accuracy using k-d tree data structures. The k-d tree search methods have a time complexity of O(log x(N)), where x is the number of branches at each node and N is the number of entries in the reference data set, which means larger reference datasets can be used to efficiently estimate each event's interaction position. The accuracy of methods described herein was found to be equal to the exhaustive search method, yielding the highest achievable accuracy. Most importantly, the disclosed methods have no restriction on the data structure of the reference dataset and can work with complicated mean detector response functions (MDRFs), meaning it is more robust compared with other methods such as contracting grid (CG) search or vector search (VS) methods.

### STATEMENT REGARDING FEDERALLY SPONSORED RESEARCH OR DEVELOPMENT

This invention was made with government support under Grant Nos. P41-EB002035 and 5R01EB000803, awarded by NIH/NIBIB. The U.S. government has certain rights in this invention.

### BACKGROUND

Scintillator-based detectors have been extensively used in both clinical and pre-clinical single photon emission computed tomography (SPECT) and positron emission tomography (PET) scanners, due to their relatively low cost, high gamma-ray stopping power, and fast timing (T. K. Lewellen, Physics in Medicine & Biology, 2008, 53(17): R287). Position-estimation algorithms or decoding methods are applied for gamma-ray interaction position localization with signals induced by scintillation photons impinging on light sensors, such as photomultiplier tubes (PMTs), silicon photomultipliers (SiPMs), etc. Anger arithmetic has dominated in gamma-ray interaction position estimation for decades due to its simplicity and fast estimation/decoding speed (H. O. Anger, Review of scientific instruments, 1958, 29(1): 27-33).

However, driven by the demand for better spatial resolution and lower bias, many other gamma-ray position estimation/decoding algorithms based on reference datasets have been proposed and studied, such as look-up table (LUT) (Aarsvold et al., Nuclear Science Symposium and Medical Imaging Conference Record, 1995, IEEE, 3: 1811), contracting-grid search (Hesterman et al., IEEE transactions on nuclear science, 2010, 57(3): 1077-1084), k-nearest neighbor (van Dam et al., IEEE Transactions on Nuclear Science, 2011, 58(5): 2139-2147), and neural network (Yonggang et al., IEEE Transactions on Nuclear Science, 2011, 58(1): 34-42). These reference-data based methods not only provide better positioning capability for gamma-ray interactions, especially at detector edges and corners, but can also filter out events below a lower merit threshold (for example, likelihood (Barrett et al., IEEE Transactions on Nuclear Science, 2009, 56(3): 725-735)) to reject events such as those that Compton scatter and deposit energy in multiple positions in the detector.

Compared with Anger arithmetic, reference-data-based methods need detector calibration to acquire their reference datasets, and the position estimation speed remains relatively slow due to the excessive computations required. Thus, the current need for position estimation is to try to achieve both high levels of accuracy and efficiency at the same time. The methods provided herein address these problems by providing systems and methods for estimating gamma-, X-, neutron, or proton-ray, or other high energy particle, interaction positions that are faster, more accurate, and more computationally and memory-use efficient than known systems and methods used in the medical imaging field.

### SUMMARY OF THE INVENTION

The present invention provides methods and systems for estimating gamma-ray interaction-positions using k-d tree searching. As applied to gamma-, X-, neutron, or proton-ray, or other high energy particle, interaction position estimation, the disclosed method can be combined with various kinds of closeness metrics such Euclidean distance, maximum-likelihood estimation, etc. Compared with conventionally known methods, the methods disclosed herein achieve high levels of speed and accuracy at the same time by using k-d tree data structures.

The k-d tree is a search tree in which every leaf is a k-dimensional point. Every non-leaf node generates at least one hyperplane that divides the space into at least two parts, generally known as half-spaces. For example, in embodiments where the node is separated into two parts by a hyperplane, points to the left of this hyperplane (such as on a number plane) are represented by the left subtree of that node and points to the right of the hyperplane are represented by the right subtree. Every node in the tree is associated with at least one of the k dimensions, with the hyperplane perpendicular to that dimension's axis (see J. L. Bentley, Communications of the ACM, 1975, 18(9): 509-517).

In an embodiment of the invention, the k-d tree search methods have a time complexity of O(logₓ(N)), where x is the number of branches at each node, and N is the number of entries in the reference data set, which means larger reference datasets are able to be used to efficiently estimate the interaction position of each event. In an embodiment, the k-d tree search methods have a time complexity of O(log₂(N)). However, it should be noted that x is not restricted to 2. If a tree is constructed with 3 branches at each node, the time complexity would be O(log₃(N)), and more branches are able to be made at each node. The accuracy of embodiments of the present invention is equal to the accuracy found in exhaustive search methods, which yield the highest achievable accuracy.

In aspects of the present invention, the methods have no restriction on the data structure of the reference dataset and are able to still work with complicated mean detector response functions (MDRFs), meaning the disclosed methods are more robust compared with other methods such as contracting grid (CG) search or vector search (VS) methods that could yield locally optimal, instead of globally optimal, results.

In an embodiment, the present invention provides a method for estimating a position of an interaction event using signals induced by scintillation photons on one or more light sensors, the method comprising:
receiving, from the light sensors and by a processor communicatively coupled to the light sensors, a dataset comprising values corresponding to the signals induced by the scintillation photons;
assigning, by the processor, a partition criteria to a k-d tree data structure having nodes and leafs, wherein the nodes include a root node, intermediate nodes, and penultimate nodes, wherein the root node, the intermediate nodes, the penultimate nodes, and the leafs are linked by branches;
storing, by the processor and in a memory device communicatively coupled to the processor, the received dataset to the k-d tree data structure;
querying, by the processor, the k-d tree for the position of the interaction event, wherein the querying step comprises: (a) calculating a compound value from a query vector; (b) determining, by the processor, a branch of the k-d tree to search based on a searching strategy; (c) searching, by the processor, the branch of the k-d tree selected by the searching strategy;
for the intermediate nodes and the penultimate nodes, iterating, by the processor, through steps (a), (b), and (c); and
returning, by the processor, the value stored in the leaf as a query result for the position of the gamma-ray interaction position.

In an embodiment, the interaction event is a gamma-ray, X-ray, neutron, proton, or other high energy particle interaction within the detector. Preferably, the interaction event is a gamma-ray and/or X-ray interaction within the detector. Optionally, the method further comprises constructing, by the processor, the k-d tree data structure as a binary search tree structure.

In an embodiment, the compound value is a value of a certain dimension of the query vector, or a linear/non-linear combination of values from two or even more dimensions of the query vector (for instance, distance to a hyperplane). Additionally, or instead, the compound value is a likelihood value, or optionally a likelihood reduction value.

Optionally, the received dataset as described herein comprise an array of values corresponding to the signals induced by the scintillation photons, and the method further comprises constructing, by the processor, the k-d tree data structure from the array of values as a one-dimensional k-d tree.

Alternatively, the received dataset comprises a plurality of vectors, each of the plurality of vectors having two or more values corresponding to the signals induced by the scintillation photons, and the method further comprises constructing, by the processor, the k-d tree data structure from the plurality of vectors as a k-d tree having at least two-dimensions.

In an embodiment, the method further comprises: determining, by the processor and based on the received dataset, mean detector response functions (MDRFs), where the MDRFs are defined as the mean signal responses of the light sensors as functions of known interaction positions (such as gamma-ray, X-ray, neutron, proton and/or other high energy particle interactions); and constructing, by the processor, the k-d tree data structure from the MDRFs.

In an embodiment, the assigning step comprises applying, by the processor, a rule for each of the nodes, wherein the querying step includes: comparing, by the processor, a compound value calculated from a query for the querying step with a boundary value stored in the root node; and selecting, by the processor and based on a result of the comparing step and the applied rule, one of the branches to search for the searching step, wherein the iterating step includes applying, by the processor, the same rule for the intermediate nodes and the penultimate nodes.

Optionally, the one or more boundary values include at least two boundary values, and comparing the compound value calculated from the query vector for the querying step with one or more boundary values stored in the root node comprises determining that the compound value falls in an interval bounded by the at least two boundary values, determining that the compound value is less than the at least two boundary values, and/or determining that the compound value is greater than the at least two boundary values.

In an embodiment, the method further comprises computing, by the processor, a distance (or merit) between the leaf storing the value of the query result and the query for the querying step.

Optionally, the receiving step includes receiving, by the processor, the dataset from light sensors of a single-photon emission computed tomography (SPECT) scanner or a positron emission tomography (PET) scanner.

Optionally, querying the k-d tree for the position of the interaction event position comprises: (a) determining, by the processor, a branch of the k-d tree that satisfies a selected searching strategy; (b) searching, by the processor, the branch of the k-d tree that satisfies the searching strategy; and (c) determining, by the processor, a value stored in the node corresponding to a result of the searching step.

In an embodiment, the present invention provides a system for estimating a position of an interaction event, the system comprising: light sensors for generating signals induced by scintillation photons incident thereupon; a memory device; and a processor communicatively coupled to the light sensors and to the memory device. The processor is programmed to:
receive, from the light sensors, a dataset including values corresponding to the signals induced by the scintillation photons;
assign a partition criteria to a k-d tree data structure having nodes and leafs, wherein the nodes include a root node, intermediate nodes, and penultimate nodes, wherein the root node, the intermediate nodes, the penultimate nodes, and the leafs are linked by branches;
store, in the memory, the received dataset to the k-d tree data structure;
querying, by the processor, the k-d tree for the position of the interaction event position, wherein the querying step comprises: (a) calculate a compound value from a query vector; (b) determine a branch of the k-d tree to search based on a searching strategy; (c) search the branch of the k-d tree selected by the searching strategy;
for the intermediate nodes and the penultimate nodes, iterate through processor operations (a), (b), and (c); and
return the value stored in the leaf as a query result for the position of the gamma-ray interaction position.

In an embodiment, the interaction event is a gamma-ray, X-ray, neutron, proton, or other high energy particle interaction within the detector. Preferably, the interaction event is a gamma-ray and/or X-ray interaction within the detector.

Optionally, the processor is further programmed to construct the k-d tree data structure as a binary search tree structure. In an embodiment, the processor is further programmed to: determine, based on the received dataset, mean detector response functions (MDRFs), the MDRFs defined as the mean signal responses of the light sensors as functions of known interaction event positions; and construct the k-d tree data structure from the MDRFs.

Optionally, the received dataset as described herein comprises an array of values corresponding to the signals induced by the scintillation photons, and the method further comprises constructing, by the processor, the k-d tree data structure from the array of values as a one-dimensional k-d tree.

Alternatively, the received dataset comprises a plurality of vectors, each of the plurality of vectors having two or more values corresponding to the signals induced by the scintillation photons, and the method further comprises constructing, by the processor, the k-d tree data structure from the plurality of vectors as a k-d tree having at least two-dimensions.

In an embodiment, for the assigning processor operation, the processor is further programmed to apply a rule for each of the nodes, and wherein, for the querying processor operation, the processor is further programmed to:
compare a compound value from a query for the querying step with a boundary value stored in the root node; and
select, based on a result of the comparing processor operation and the applied rule, one of two branches to search for the searching processor operation,
wherein, for the iterating processor operation, the processor is further programmed to apply the same rule for the intermediate nodes and the penultimate nodes

In an embodiment, the processor is further programmed to compute a distance (or merit) between the leaf storing the value of the query result and the query for the querying processor operation.

Optionally, the system comprises a medical imaging system including the light sensors, wherein, for the receiving processor operation, the processor is further programmed to receive the dataset from light sensors of medical imaging system. Such medical imaging systems include, but are not limited to, a single-photon emission computed tomography (SPECT) scanner, and a positron emission tomography (PET) scanner.

In an embodiment, the present invention provides a non-transient computer readable medium comprising processor-executable instructions stored therein for estimating a position of an interaction event, which, when executed by one or more processors communicatively coupled to one or more memory devices, and light sensors for generating signals induced by scintillation photons incident thereupon, cause the one or more processors to:
receive, from the light sensors, a dataset including values corresponding to the signals induced by the scintillation photons;
assign a partition criteria to a k-d tree data structure having nodes and leafs, wherein the nodes include a root node, intermediate nodes, and penultimate nodes, wherein the root node, the intermediate nodes, the penultimate nodes, and the leafs are linked by branches;
store, in the memory, the received dataset to the k-d tree data structure;
query the k-d tree for the position of the interaction event position, wherein, for querying the k-d tree, the processor-executable program instructs the one or more processors to: (a) calculate a compound value from a query vector; (b) determine a branch of the k-d tree to search based on a searching strategy; (c) search the branch of the k-d tree selected by the searching strategy;
for the intermediate nodes and the penultimate nodes, iterate through the processor operations (a), (b), and (c); and
return the value stored in the leaf as a query result for the position of the gamma-ray interaction position.

In an embodiment, the interaction event is a gamma-ray, X-ray, neutron, proton, or other high energy particle interaction within the detector. Preferably, the interaction event is a gamma-ray or X-ray interaction within the detector.

Representative claims are provided herein, and are specifically incorporated by reference.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

The figures interspersed throughout the application are specifically incorporated by reference herein.

FIG. 1 shows a 1-d tree in an embodiment of the invention, sorting the 8 numbers in ascending order. The ellipses are nodes and boxes are leaves.

FIG. 2 shows an example of a 2-d tree organizing 16 2-d vectors. Each leaf in the tree represents a 2-d vector.

FIG. 3 shows a 2-d map representing the 16 2-d vectors of FIG. 2. Each dot is one vector.

FIG. 4 shows the 2-d map of FIG. 3 segmented by the 2-d tree. Each line represents a node in the 2-d tree.

FIG. 5 shows a 3-d tree segmenting the 3-d space into multiple subspaces.

FIG. 6 shows MDRFs achieved by a simulation of an ideally thin gamma-ray beam of 511 keV gamma-ray photons.

FIG. 7 shows an estimated projection image of seven fan beams of width 0.44 mm, using an exhaustive search method. Variance is large near detector edges due to the undesirable total internal reflection (TIR) on detector edges.

FIG. 8 shows an estimated projection image of the seven fan beams of width 0:44 mm, using a contracting-grid search method.

FIG. 9 shows a likelihood map of three random events.

FIG. 10 shows an estimated projection image of the seven fan beams using a k-d tree search method.

FIG. 11 shows measured MDRFs of a prototype detector in an embodiment of the invention.

FIG. 12 shows six slit projection images estimated using the exhaustive search method.

FIG. 13 shows the six slit projection images estimated using the contracting-grid search method.

FIG. 14 shows the six slit projection images estimated using the k-d tree search method.

FIG. 15 shows a schematic diagram of a system for estimating a position of an interaction event which generates scintillation photons within a detector in an embodiment of the invention.

FIG. 16 shows a flow chart of a method for estimating a position of an interaction event which generates scintillation photons within a detector using signals induced within a detector by scintillation photons on one or more light sensors in an embodiment of the invention.

### DETAILED DESCRIPTION OF THE INVENTION

In general, the terms and phrases used herein have their art-recognized meaning, which can be found by reference to standard texts, journal references and contexts known to those skilled in the art. Any definitions used herein are provided to clarify their specific use in the context of the invention.

### Overview

Previous methods for estimating interaction positions have used mean-detector-response functions (MDRFs) derived from calibration to represent the characteristics of the detector, and to search among these candidate positions in the MDRFs using contracting-grid (CG) search methods to find the position with highest likelihood compared to other candidates (ML) (Hesterman et al., IEEE Transactions on Nuclear Science, 2010, 57(3): 1077-1084). However, these methods may fail to find the global maximum with detectors having complicated MDRFs, such as edge-readout detectors with optical barriers (Li et al., Medical physics, 2018, 45(6): 2425-2438). As described in embodiments herein, methods are provided that can accurately estimate gamma-, X-, neutron, or proton-ray, or other high energy particle, interaction position with satisfactory accuracy and with a short search time. In embodiments of the present invention, these methods use a multi-dimensional tree structure (k-d tree) (J. L. Bentley, Communications of the ACM, 1975, 18(9): 509-517) constructed with mean-detector-response functions (MDRFs) to assist finding the nearest neighbor of the gamma-, X-, neutron, or proton-ray, or other high energy particle, interaction event signal among the candidates in the MDRFs.

Generally, speaking, in the process of searching results for a query vector, at each node, a compound value is calculated by a series of linear/non-linear combinations of values from the query vector: For example, the compound value may equal v[0]+2v[1]+0.03*(v[2])².

Each node in the k-d tree contains a series of boundary values (1 boundary value for a 2 branch case, 2 boundary values for a 3 branch case, 3 boundary values for a 4 branch case, etc.). The searching process involves comparing the compound value with the boundary value, and finding the branch that satisfies a selected searching strategy that corresponds to the partition criteria (i.e., determine if the compound value falls within, above or below an interval bounded by the boundary values).

The searching is continued in the branch that satisfies the searching strategy. Additionally, searching can also be continued in the branch that violates the searching criteria (in this case, accumulated error increases).

A search is continued in a branch if the accumulated error is still smaller than the distance (or merit) between the query vector and current “Champion” leaf. If the accumulated error is larger than the distance (or merit), then branch will not be searched further.

These k-d tree search methods have superior accuracy since the accuracy of position estimates is equivalent to exhaustive search method. Also, utilizing the k-d tree data structure, searching is much faster and more efficient as less computations are required.

### Example 1. Brief Introduction about k-d Tree

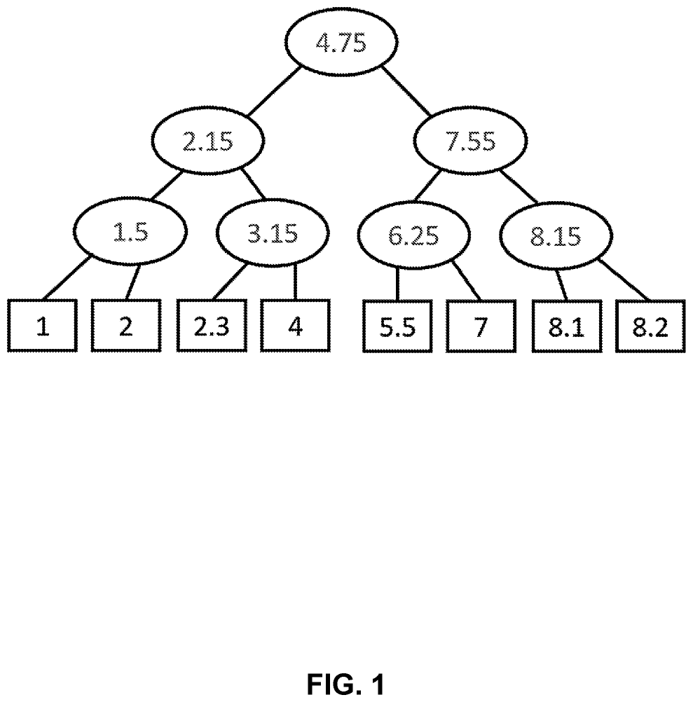

K-d tree stands for k-dimensional tree—a tree structure constructed with data of k dimensions. High dimensional hyperplanes are used to subdivide the reference data set into many partitions, which will eventually assist the searching process. A one-dimensional tree can be called 1-d tree, which is actually a binary tree and relatively easy to understand. An example of the one-dimensional case is shown in FIG. 1.

### 1.1. One-Dimensional Case

Suppose there is an array of 8 numbers: {1, 2, 2.3, 4, 5.5, 7, 8.1, 8.2}. In such a case, a 1-d tree can be constructed. The first node can be constructed with a certain value that splits the whole array of numbers into two parts. The numbers (e.g., the array elements) smaller than the value of the node's number go to the left branch of the node, while the numbers greater than the node's number go to the right branch of the node. The second and third nodes can be found in the left and right branches of the root node, respectively, using the same procedure. The nodes are constructed continuously until only one element is left in a branch, and this element is defined as a leaf. Such a 1-d tree constructed is shown in FIG. 1, where all the above 8 numbers are stored as leaves.

For the use case illustrated in FIG. 1, the value of first node of the 1-d tree was chosen to be 4.75, which is actually the mean value of 4 and 5.5 for simplicity. The second node's value is the medium of the numbers in the left branch of the first node, that is the medium of {1, 2, 2.3, 4}, which is the mean of 2 and 2.3. This procedure is repeated until all the numbers in the array become leaves, indicating that the binary search tree has been constructed. The 1-d tree can sort the numbers in either ascending or descending order, depending on the rule to construct the tree.

This 1-d tree is also a binary search tree, and searching an element can be very efficient, especially when the total number of leaves becomes large. For example, to determine if the number 4 is in the tree or not, the searching process can start with comparing 4 with the root node's value which is 4.75. Since 4<4.75, the process starts to search the node's left branch. Then, 4 is compared with 2.15 (the second node) and since 4>2.15, the process starts to search the second node's right branch. The searching continues until 4 is found in the leaf node. The total time complexity of searching a binary search tree is O(logₓ(N)), where x is the number of branches at each node (x=2 for a binary search tree), and where N is the number of total leaves in the binary search tree.

Where the number of branches at each node is 2 (the binary search tree case), the time complexity would be O(log₂(N)). However, the number of branches at each node can be greater than 2, (i.e., x can be 3, 4, 5 or more). For example, a node can have three branches where v[0]+2v[1]<−1.5 (left branch), v[0]+2v[1]>=−1.5 and v[0]+2v[1]<=2 (middle branch), and v[0]+2v[1]>2 (right branch).

### 1.2. Two-Dimensional Case

A 2-d tree is a two-dimensional binary search tree, whose leaves have two dimensions. Each node of the 2-d tree also splits the whole (e.g., current) dataset into two data subsets. Since the dataset contains 2-d vectors, it is vital to decide in which of the two dimensions to set the node's value that splits the dataset. In the following example, the splitting dimension is randomly picked to set the node's value.

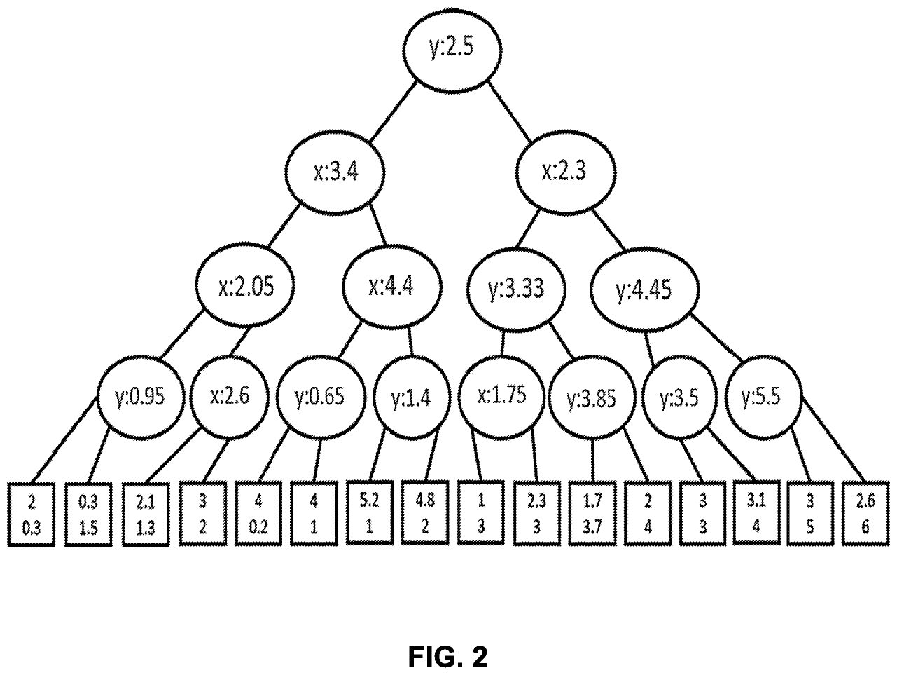

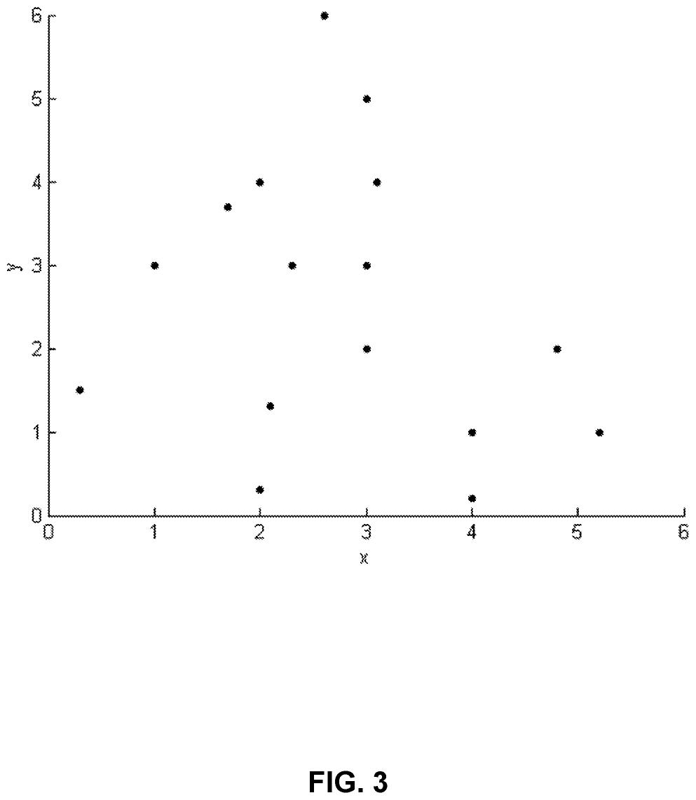

The dataset (containing 2-d vectors) to construct an example 2-d tree is: {[0.3, 1.5], [2.3, 3], [2.1, 1.3], [3, 3], [1.7; 3.7], [2; 0.3], [3; 5], [2.6, 6], [3, 2]; [3.1, 4], [1, 3]; [2, 4], [5.2, 1], [4.8, 2], [4, 0.2], [4, 1] }. The corresponding constructed 2-d tree is shown in FIG. 2, where each leaf in the tree represents a 2-d vector. The 16 2-d vectors can be plotted on a 2-d map, as shown in FIG. 3, where each dot is one vector.

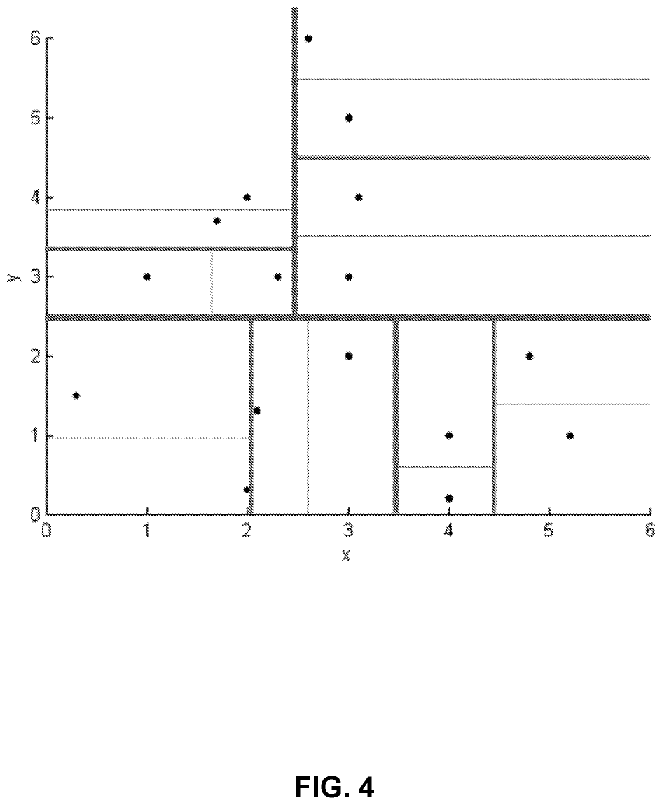

The nodes of the 2-d tree work as partitions to segment the region containing the 2-d vectors (points in the map). For example, the first node y: 2.5 is a line (y=2.5), separating 2-d vectors equally into 2 groups. The 2-d vectors whose y component is lower than 2.5 are classified into its left branch, while the 2-d vectors whose y component is larger than 2.5 are classified into its right branch. The 2-d map segmented by the 2-d tree is shown in FIG. 4, where each line represents a node in the 2-d tree.

After the construction of the 2-d tree, a search can be done similarly to the 1-d case. For example, to search whether a 2-d vector [1, 3] is in the tree or not, the y component value 3 of this vector is compared with 2.5; since 3>2.5, the search should continue in the right branch. Then, the x component value 1 of this vector is compared with 2.3; since 1<2.3, the subsequent search should be done in the left branch. Then 3 is compared with 3.3; since 3<3.3, the search should continue to the left branch. Then 1 is compared with 1.75; since 1<1.75, and the left branch is a leaf, the vector [1, 3] is compared with this leaf. Because they are equal, it can be concluded that the vector [1, 3] is in this 2-d tree.

### 1.3. Multi-Dimensional Tree

A k-d tree is a binary tree based on data vectors with k dimensions. Similar to the 2-d tree case, each node has two attributes. The first attribute is the index of dimension, and the second attribute is the value indicating the boundary for partition. The index of dimensionality indicates on which axis the partition occurs, and the partition value indicates the specific partition position on the axis.

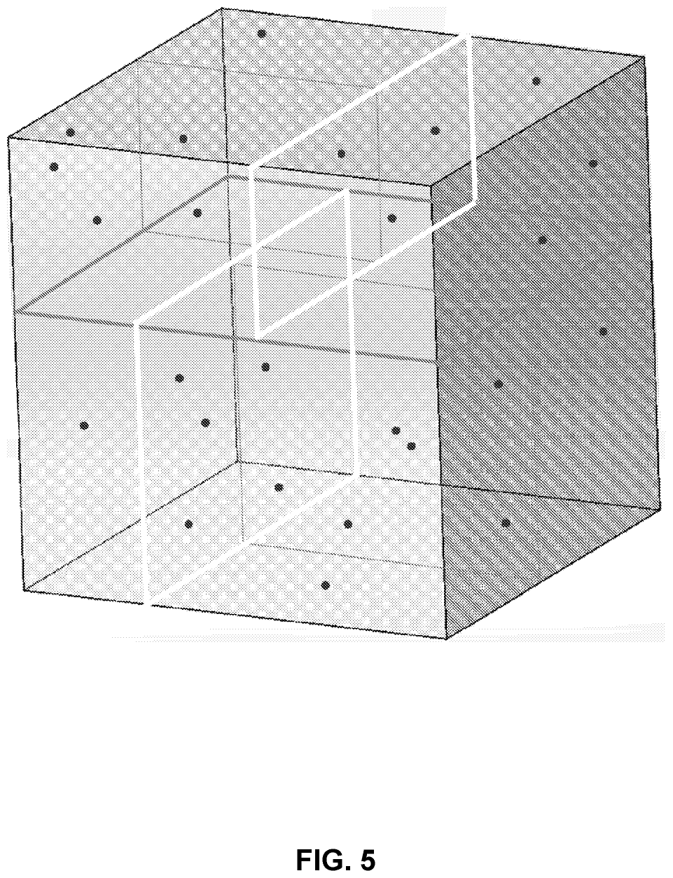

A 3-d tree can be viewed as a series of planes segmenting the 3-d space into multiple subspaces, that contains data points as equally as possible, as shown in FIG. 5 (WIKIPEDIA, “Example of 3d tree,” en.wikipedia.org/wiki/K-d_tree [Online; accessed 19 Feb. 2018]). It may be hard to image a 4-d or 5-d tree, but the basic principle is the same.

Note that the criteria for constructing each node of the multi-dimensional tree is that: The dimension and value that “best” split the reference data (or entry data) are chosen. Since under the influence of noise, a small gap between two branches will cause crosstalk between its two branches, events could be misclassified into the wrong branch, which induces search error, which would slow down the searching process as introduced below. So, the gaps between two branches should be as large as possible, especially for the nodes close to root node.

### Example 2. Searching for k-Nearest Neighbor (KNN)

One application of the k-d tree is to search for the presence of a specific event efficiently. However, other methods such as a hash table, or maps, can do a search even more efficiently. Moreover, when the event matches no entry (leaf) of the k-d tree, one may still be interested in finding the unknown event's nearest neighbors among the entries (leaves). That is, to use certain figure-of-merit metrics to measure the distance between the event and all the entries in the k-d tree and find the k entries with the highest or lowest merit values. This problem is called the k-nearest neighbor problem. Note that the “k” here is different from the “k” in k-d tree: whereas the “k” in “k-nearest neighbor” represents the number of entries in a dataset with highest or lowest merit values, the “k” in “k-d tree” stands for the dimensionality of the events inside the tree. For purposes of this disclosure, only the “nearest” entry is searched for, which corresponds to a 1-nearest neighbor problem.

For example, if the query event (also referred to herein as “query vector”) is denoted as \vec{e}, one entry inside the k-d tree is noted as \vec{l}, and the dimensionality is M, the Lₚ norm can be used as the merit value to measure their closeness:

$$merit ( \vec{l} ) = {( {\sum }_{i = 1}^{M} {\vert {e}_{i} - {l}_{i} \vert}^{p} )}^{1 \text{/} p} \qquad ( 1 )$$

The goal is to find the entry in the reference dataset with the smallest distance to the query event. An exhaustive search can be used to solve this problem, but the relatively long searching time is a big disadvantage for processing many events. The k-d tree can be used to organize the entries in the reference dataset into a binary search tree structure across k dimensions, and the search for the nearest neighbors for the query event can then be performed efficiently.

### 2.1. Pruning of a Branch

The searching process traverses the whole tree while following a certain strategy. For example, a depth-first search strategy can be used in the algorithm. In this work, the branch that satisfies the partition criteria is always searched first. For instance, at a node, if event[node.idx]<node.value, then the left branch is searched first. The left branch stores entries so that entry[node.idx]<node.value, and node.idx indicates the dimension to split, while node.value is the boundary value. When a leaf node is visited, equation (1) can be used to calculate the distance between the event and the leaf. In this example, the p in equation (1) is set as 2, which corresponds to a Euclidean distance for simplicity. The current minimum distance (meritₘᵢₙ) and its corresponding entry values (reference vector) are stored.

However, in order to accelerate the searching process, some branches can be pruned (not searched) if certain conditions are met. A vector \vec{cost} is used to store the deviation in each dimension, so the vector \vec{cost} has the same dimension as an event vector. The deviation is caused by choosing the branch that violates the partition criteria. For example, at a node, if event[node.idx]<node.value, but the right branch is searched instead of the left branch, then the deviation (dev) is defined as |event[node.idx]−node.value|. If dev>cost[node.idx], then the deviation dev is used to update the corresponding entry of \vec{cost} (cost[node.idx]=dev). Then before searching the right branch, the total deviation devₜₒₜₐₗ=(Σᵢ₌₁^{M}cost[i]²)^{1/2} is computed. If devₜₒₜₐₗ<meritₘᵢₙ, the search can be extended to include the right branch; otherwise, there is no need to search as the accumulated error already exceeds the that of current best result, meaning no better (closer) entry can be found in the right branch.

The pruning can greatly increase the search speed, since a large fraction of branches can be excluded from the search path for a specific event. This is the key aspect of k-d tree search, since if no pruning is done, the total search time is equivalent to that of the exhaustive search method.

### 2.2. A 2-d Tree Search Example

Take the 2-d tree in FIG. 2 for example. If the query event is [4.8, 2.6], and it is desired to find its nearest neighbor in the tree, then the search process can be described as follows:

1. At root node y: 2.5, compare y components; since 2.6>2.5, go to node x: 2:3.

2. Compare the x component; since 4.8>2.3, go to node y: 4.45.

3. Compare y components; since 2.6<4.45, go to node y: 3.5.

4. Compare y components; since 2.6<3.5 and since the left branch is a leaf, compare the query event with the leaf. The distance between the query vector and this leaf is \sqrt{(4.8−3)²+(2.6−3)²}≈1.84; record this distance as meritₘᵢₙ, then return to node y: 3.5.

5. Before visiting its right branch, the total deviation needs to be calculated and compared with merit. Because devₜₒₜₐₗ=|3.5−2.6|=0.9, which is smaller than meritₘᵢₙ, =1.84, a better result (closer entry) could still be found in this node's right branch and the search is continued there.

6. Compare the query vector [4.8, 2.6] with the leaf [3.1, 4]; because the distance is 2.20, which is larger than meritₘᵢₙ, then go back to node y: 3.5.

7. Since both the branches of node y: 3.5 have been visited, continue to go back to node y: 4.45.

8. Because devₜₒₜₐₗ=|4.45−2.6|=1.95>meritₘᵢₙ=1.84, there is no need to search its right branch, then continue to go to node x: 2.3.

9. Because devₜₒₜₐₗ=|4.8−2.3|=2.5>meritₘᵢₙ=1.84, there is no need to search its left branch, then go back to node y: 2.5.

10. Because devₜₒₜₐₗ=|2.6−2.5|=0.1<meritₘᵢₙ=1.84, it is possible to find a better result in its left branch, then go to node x: 3.4.

11. Compare x components; since 4.8>3.4, go to node x: 4.4.

12. Compare x components; since 4.8>4.4, go to node y: 1.4.

13. Compare y components; since 2.6>1.4, and the right branch is a leaf, compare the query vector [4.8, 2.6] with the leaf [4.8, 2]; the distance is 0.6<meritₘᵢₙ=1.84, thus a better result is found; update meritₘᵢₙ with 0.6 and record this leaf [4.8, 2], then go back to node y: 1.4.

14. Because devₜₒₜₐₗ=|2.6−1.4|=1.2>meritₘᵢₙ=0.6, there is no need to search its left branch; go back to node x: 4.4.

15. Since devₜₒₜₐₗ=\sqrt{(4.8−4.4)²+(2.6−2.5)²} ≈0.412 (considering cumulative error in both x and y dimensions, as there is already 0.1 error in y dimension)<meritₘᵢₙ=0.6, a better result might be found in its left branch; go to node y: 0.65.

16. Compare y components; since 2.6>0.65, compare the query vector with its right leaf; because the distance is 1.79>meritₘᵢₙ=0.6, go back to node y: 0.65.

17. Because devₜₒₜₐₗ=\sqrt{(4.8−4.4)²+(2.6−0.65)²}≈1.99>meritₘᵢₙ=0.6, there is no need to search the left branch; go back to node x: 4.4.

18. Since both branches of the node x: 4.4 have been searched, continue to go back to node x: 3.4.

19. Since devₜₒₜₐₗ=\sqrt{(4.8−3.4)²+(2.6−2.5)²}≈1.40>meritₘᵢₙ=0.6, there is no need to search in its left branch; go back to node y: 2.5.

20. Both branches of the root node y: 2.5 have been searched, the whole searching is finished; the nearest neighbor is vector [4.8, 2].

In summary, different actions are taken according to different conditions:

Condition 1: If none of the current node's two branches have been searched, then search the branch that satisfies the partitioning criteria with the query event.

Condition 2: If one of the current node's two branches has already been searched, then determine whether the other branch satisfies devₜₒₜₐₗ<meritₘᵢₙ; if so, search in the other branch; if not, go to its parent node.

Condition 3: If both of the node's two branches are searched, then go back to its parent node.

Condition 4: If this node is actually a leaf, compute its distance to the query event. If this distance is smaller than the current meritₘᵢₙ value, then update the meritₘᵢₙ and record the leaf's position.

Note: The meritₘᵢₙ is initialized with a very large number. The process of search is terminated if all branches of the root node have been searched or pruned.

2.3. Integrating Maximum-Likelihood Estimation into k-d Tree Search

The example above uses the Euclidean distance as the metric to judge the closeness of a query vector to a reference data sample. Other metrics that incorporate statistic properties of the data, such as likelihood, can also be used with k-d tree search. If the noise characteristics of the detector can be modeled accurately, the maximum likelihood method will yield more accurate results (Barrett et al., IEEE Transactions on Nuclear Science, 2009, 56(3): 725-735).

If the numbers of detected photons of a single gamma-ray interaction on the k light sensors are n₀, n₁, . . . n_{K-1}, and if a reference sample's values in a leaf are r₀, r₁, . . . r_{K-1}, then under assumption of Poisson statistics, the probability of the detected photons given the reference sample is:

$$P ( {n}_{0} , {n}_{1} , … , {n}_{K - 1} \vert {r}_{0} , {r}_{1} , … , {r}_{K - 1} ) = {\prod }_{i = 0}^{K - 1} \frac{{e}^{- {r}_{i}} {r}_{i}^{{n}_{i}}}{{n}_{i} !} \qquad ( 2 )$$

After taking the logarithm of both sides:

$$ln ( P ( {n}_{0} , {n}_{1} , … , {n}_{K - 1} \vert {r}_{0} , {r}_{1} , … , {r}_{K - 1} ) ) = {\sum }_{i = 0}^{K - 1} - {r}_{i} + {n}_{i} ln ( {r}_{i} ) - ln ( {n}_{i} ! ) \qquad ( 3 )$$

Dropping the constant term ln(nᵢ!), which does not change with rᵢ we define:

$$L ( {n}_{0} , {n}_{1} , … , {n}_{K - 1} \vert {r}_{0} , {r}_{1} , … , {r}_{K - 1} ) = {\sum }_{i = 0}^{K - 1} - {r}_{i} + {n}_{i} ln ( {r}_{i} ) \qquad ( 4 )$$

Therefore, in order to find the nearest neighbor in terms of likelihood, we need to find the leaf with the set of r₀, r₁, . . . r_{K-1}, that maximize equation (4). For each dimension (or light sensor) i, the corresponding Lᵢ is:

$${L}_{i} ( {n}_{i} , {r}_{i} ) = - {r}_{i} + {n}_{i} ln ( {r}_{i} ) \\ and L ( {n}_{0} , {n}_{1} , … , {n}_{K - 1} \vert {r}_{0} , {r}_{1} , … , {r}_{K - 1} ) = {\sum }_{i = 0}^{K - 1} {L}_{i} ( {n}_{i} , {r}_{i} ) \qquad ( 5 )$$

According to equation (5), at a node of the k-d tree, whose boundary value is bᵢ if we choose the branch that satisfies the partition criteria (if nᵢ>bᵢ, we search among rᵢ that satisfy rᵢ>bᵢ; if nᵢ<bᵢ, we search among rᵢ that satisfy rᵢ<bᵢ), it is easy to show that the highest possible Lᵢ is achieved when rᵢ=nᵢ:

Lᵢ_{ₘₐₓ}=−nᵢ+nᵢ ln(nᵢ) (6)

If we choose the branch that violates the partition criteria, the highest Lᵢ possible is achieved when rᵢ=bᵢ:

Lᵢ_{ₘₐₓ}=−bᵢ+nᵢ ln(bᵢ) (7)

In this case, if we already have a candidate result, whose L is L_{current}, the pruning can happen when a search has violated the partition criteria at a number of dimensions, such that their corresponding Lᵢₘₐₓ are limited by equation (7). For the remaining dimensions, even if their corresponding Lᵢₘₐₓ have achieved the highest values indicated by equation (6), the total L is still smaller than L_{current}, so there is no need to continue to search the branch (pruned).

This part shows the possibility of combining other metrics, such as maximum likelihood, into the k-d tree search method, which expands the flexibility of the algorithm.

### Example 3. Using k-d Tree to Estimate Event Position

Mean detector response functions (MDRFs) are the mean signal responses of the light sensors of a scintillator detector, as functions of the gamma-, X-, neutron, or proton-ray, or other high energy particle, interaction positions, and they can be treated as maps of signal responses (of the light sensors). The position of an unknown event is found by finding among the many MDRF samples the position (e.g., the “champion”) that has the smallest distance with respect to the query event (nearest neighbor). The number of total light sensors (M) is equal to the dimensionality of each event, the number of candidate positions (A in MDRFs corresponding to the number of entries of the k-d tree. Therefore, a k-d tree can be constructed with the MDRFs naturally.

In order to account for the energy variations induced by scintillator's intrinsic energy resolution, such as Poisson statistics and electric noise, each event could be normalized by the summed signal value, before searching for its nearest neighbor in the k-d tree. If doing so, the MDRFs samples used to construct the k-d tree also need to be normalized.

The k-d tree was constructed by continuously subdividing the MDRFs' samples in k-dimensional space. The dimension to subdivide the MDRF samples was chosen randomly for purposes of this disclosure, and the boundary of the subdivision was chosen as the medium of the set of MDRF-sample values corresponding to that dimension.

### 3.1. Simulation

To illustrate the utility of a k-d tree search in the determination of gamma-ray, interaction location, a simulation was carried out based on the edge-readout detector design (Li et al., Medical physics, 2018, 45(6): 2425-2438). The size of the LYSO scintillator was chosen to be 50:8 mm×50:8 mm×3 mm, 20 SiPMs were modeled, each SiPM size was 10:16 mm×3 mm, with an active area of 10 mm×3 mm. The photon detection efficiency (PDE) was conservatively set to be 20% and the dark count rate was modeled as 1.0 MHz/mm². An excess noise factor of 1.21 was included. Refractive index of the optical gel and the SiPM window material were set to be 1:5. Optical barriers were included in the crystal in accordance with Li et al., Medical physics, 2018, 45(6): 2425-2438.

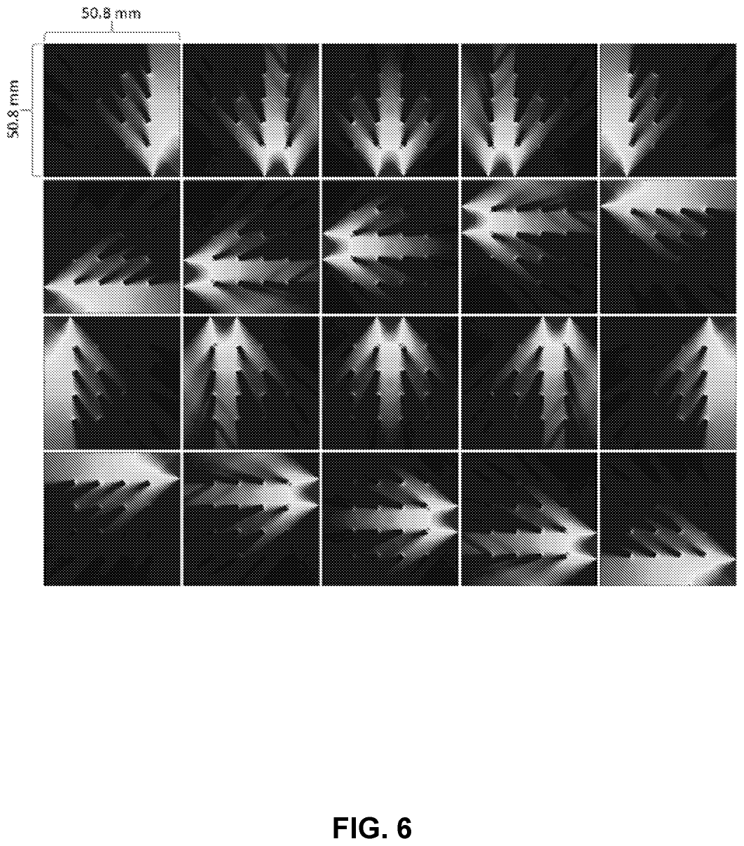

A perfectly thin gamma-ray beam of 511 keV photons was used to calibrate the detector at 252×252 positions, with step sizes in x and y directions of 0.25 mm. 1000 photoelectric interactions were modeled each scanned position. The resulting MDRFs were determined by calculating the mean signal responses of each light sensor at each scanned location, as shown in FIG. 6.

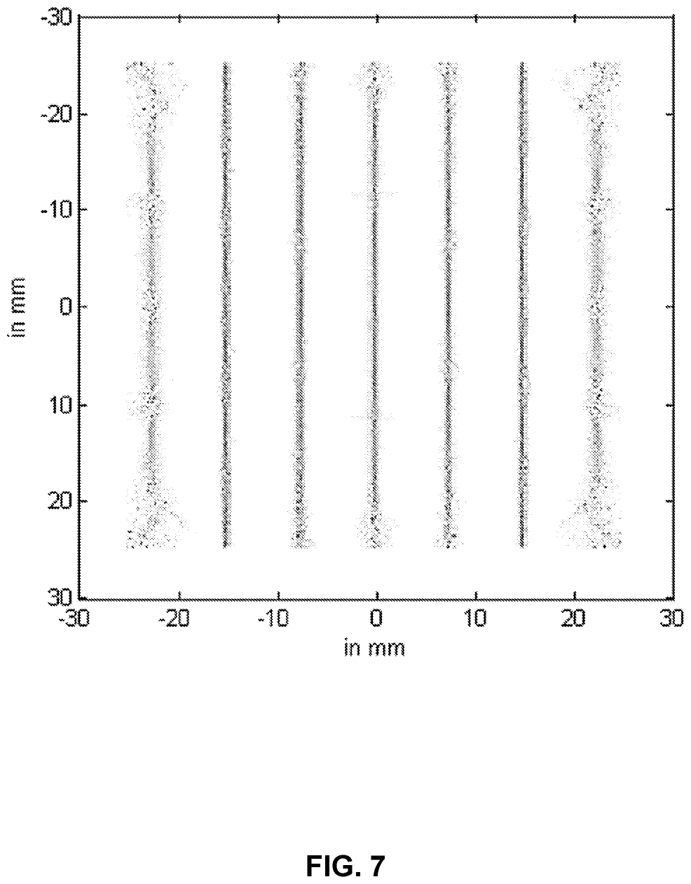

Projections created by illumination of 511 keV photons through a collimating mask with 7 parallel slits (7 fan beams of gamma rays) were also simulated. The spacing between the beams was 7.5 mm and the width of each gamma-ray fan beam was 0.44 mm FWHM. The estimated projection image of these seven fan beams on the detector is shown in FIG. 7, with a total of 9.3×10⁵ photoelectric interaction events estimated using an exhaustive search method, and where variance is larger near detector edges partially due to undesirable total internal reflection (TIR) on detector edges (Li et al., Medical physics, 2018, 45(6): 2425-2438). This result serves as the ground truth. The simulation and searching programs were written in C++.

### 3.1.1. Contracting-Grid Search Method

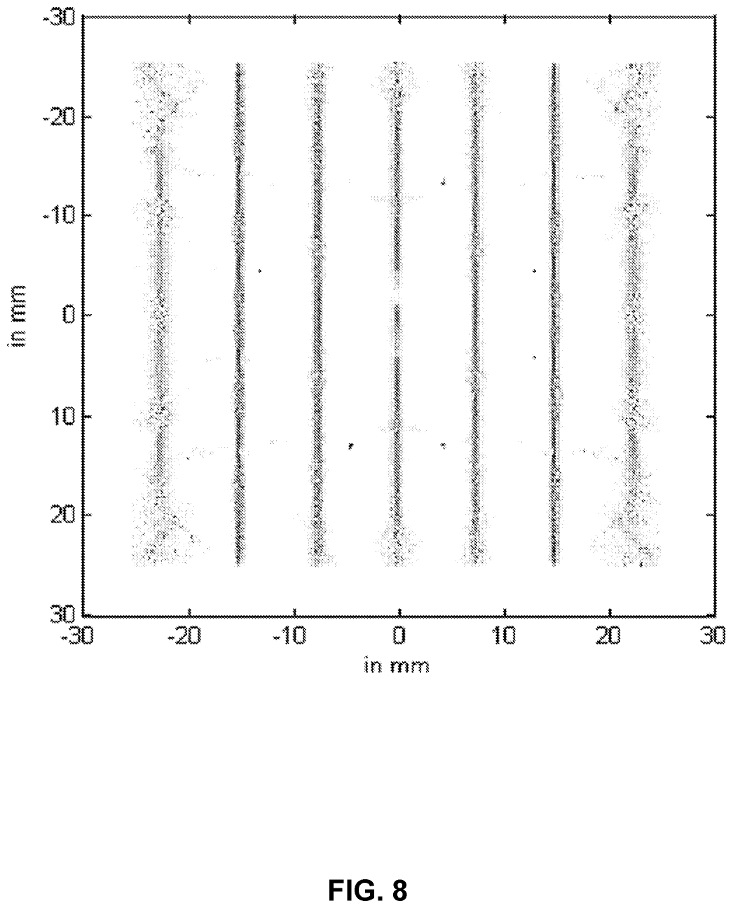

The total time of using the exhaustive search to estimate the interaction positions of the 9.3×10⁵ photoelectric interactions was 4292 s, with an Intel i7-4770k CPU, single thread, without over clocking. Using the contracting-grid (CG) search method with a 4×4 grid with initial length of 16 mm and a contracting ratio of 0.75, the total time cost is greatly reduced to 26 s, and the resulting estimated projection image for 7 fan beams is shown in FIG. 8.

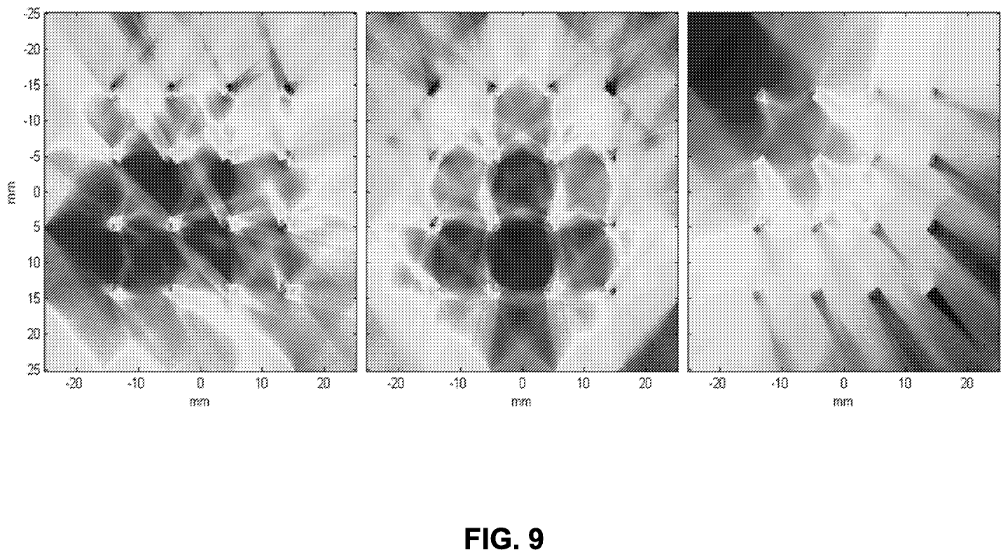

By comparing FIG. 8 with FIG. 7, it is obvious that the contracting grid (CG) search method cannot provide enough precision with these complicated MDRF function. Positioning errors are obvious in the image of FIG. 8 as the beam in the center is disconnected, and some events are misplaced in some wrong places. Compared with the ground truth, the standard deviation is 1.9 mm. The reason why the contracting grid search method does not work well is due to the introduction of optical barriers, which make the MDRFs much more complicated shapes, and there are many local-optimal positions (local likelihood maxima). The searching grid can be easily trapped into local-optimal positions and will thus at least occasionally fail to reach the global-optimal position (global likelihood maxima). A likelihood map of three random interaction events is shown in FIG. 9.

To search for a global likelihood maxima is extremely challenging due to the appearance of many local likelihood maxima. From the likelihood map of FIG. 9, many local likelihood maxima can be observed. These local likelihood maxima have chance to trap the search grid into the regions close to those local likelihood maxima and the search grid will never reach the position of global likelihood maxima.

### 3.1.2. k-d Tree Search Method

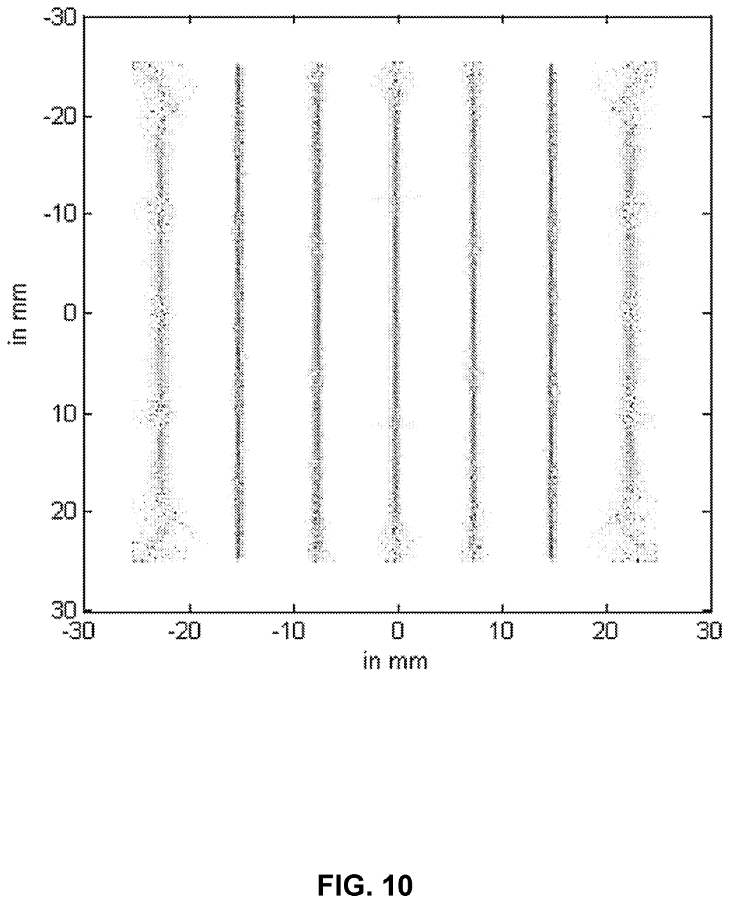

The k-d tree was constructed with the MDRFs achieved from t calibration simulation procedure described above in 3.1. In this case, each entry in the k-d tree has 20 dimensions (k is 20 in this example, since there were 20 light sensors), and there are a total of 252×252 MDRF samples (corresponding to 63,504 leaves of the tree). The interaction positions of the 7 simulated fan beams were estimated using the k-d tree search method introduced in the previous section. The resulting projection image is shown in FIG. 10.

Using the k-d tree search method, the total time used to search these 9.3×10⁵ events is 14 s, which is faster than the contracting-grid search method. By comparing FIG. 7 and FIG. 10, the precision of k-d tree search method is comparable to that of the exhaustive search method. This is not unexpected since the k-d tree search is actually another kind of exhaustive search, but with branch pruning to increase the searching speed.

### 3.2. Experiment Result

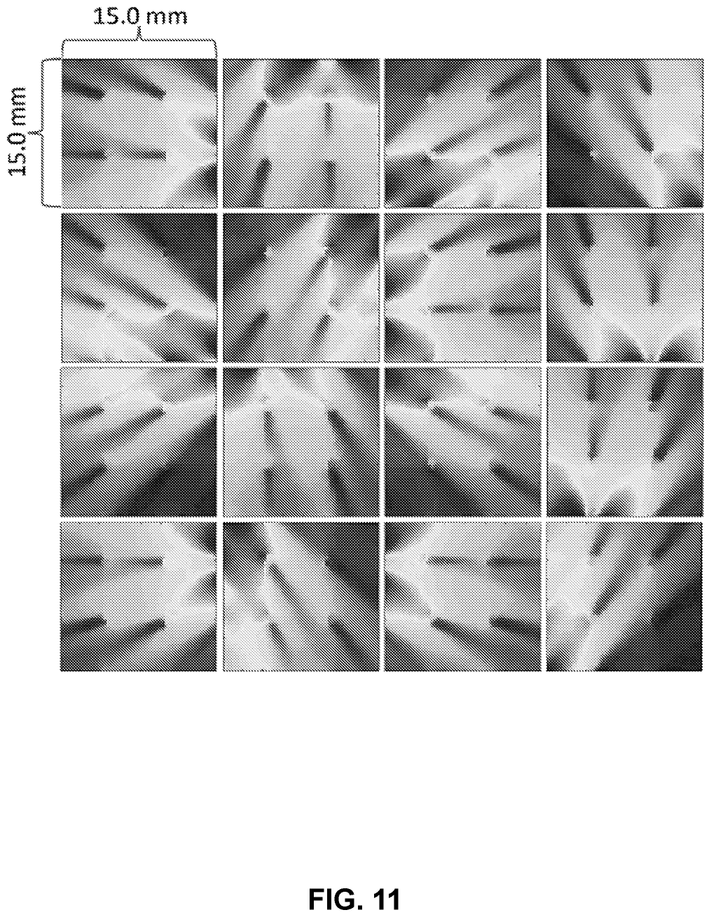

The prototype detector in Li et al. (Medical physics, 2018, 45(6): 2425-2438) was used for the experiment measurements, the scintillation crystal is a piece of Csl(T1) of size 27.4 mm×27.4 mm×3 mm, 16 Hamamatsu S13360-6050PE SiPMs (active area is 6 mm×6 mm) were attached to the scintillator crystal to read from edges (4 SiPM sensors on each edge). Sixteen mechanically drilled holes were introduced as optical barriers. The detector was calibrated using a well collimated gamma-ray beam of 662 keV, the size of the calibration beam was 0.44 mm×0.44 mm FWHM, which was used to scan 81×81 locations with a step size of 0.25 mm. The corresponding MDRFs are calculated using the calibration data, and are shown in FIG. 11. A k-d tree is constructed with this set of MDRFs, the k-d tree is constructed by continuously splitting the MDRFs' entries in k-dimensional space (k=16 in this experiment). In the process of constructing the k-d tree, the dimension which has largest gap across the split value region is always chosen, aiming to minimize the error chance of initial search.

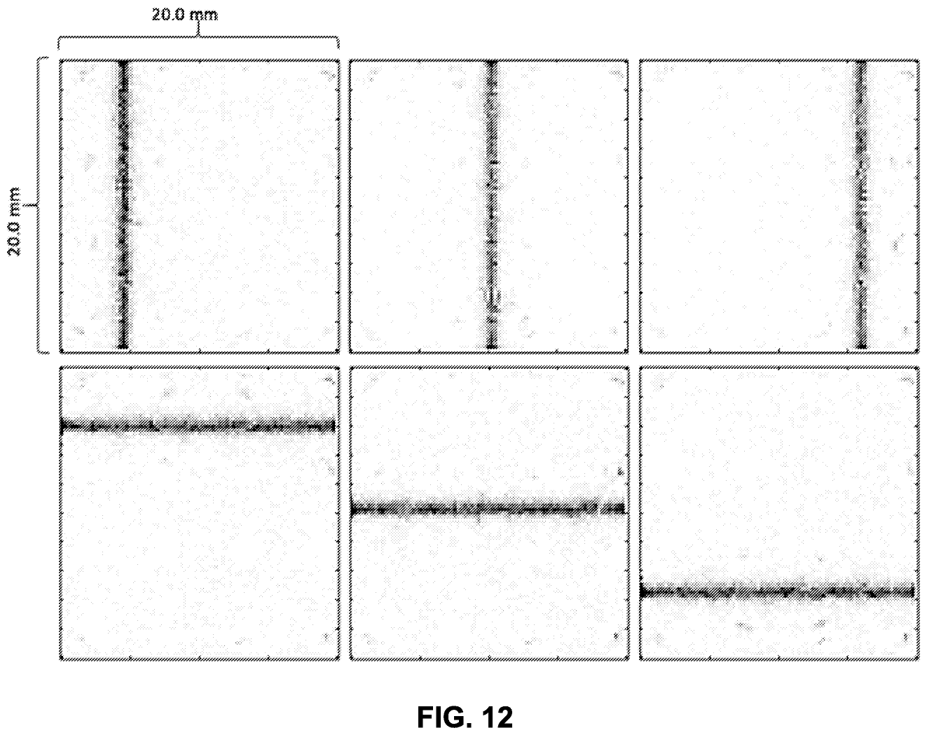

To test the positioning algorithm, six slit beams of width 0.625 mmFWHM at six different positions were projected onto the detector, 50000 gamma-ray interactions of 662 keV photons were recorded at each slit location. FIG. 12 shows the estimated projection images of the 6 slit beams using exhaustive search method, which are recorded, and considered, as the ground truth. It took 162 s to finish searching the 3×10⁵ events.

In the following part, a comparison was made between contracting-grid (CG) search method and k-d tree search method.

### 3.2.1. Contracting-Grid Search

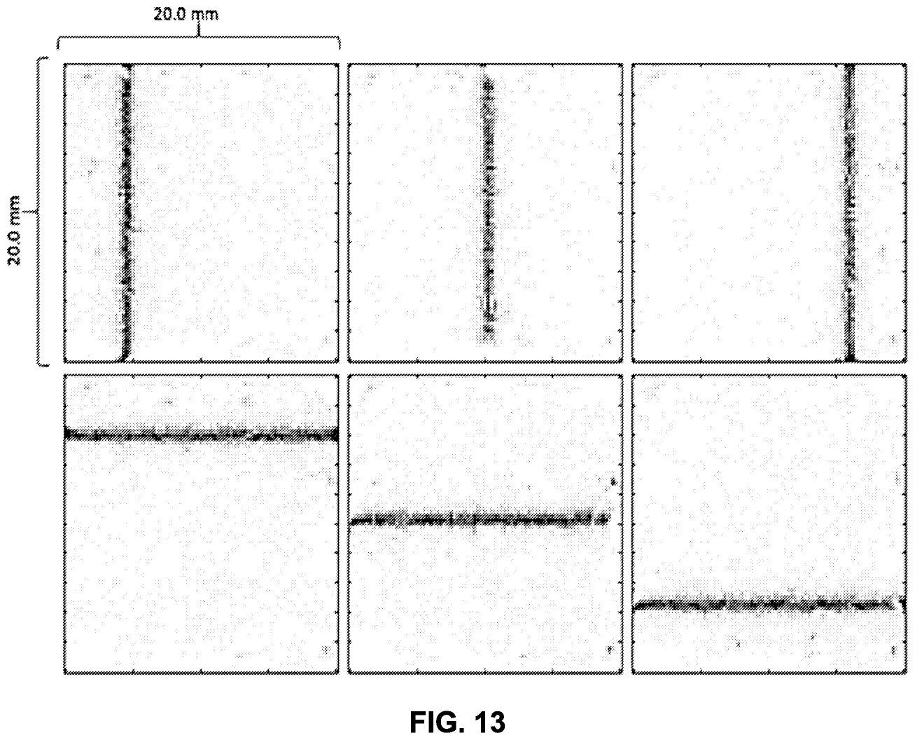

The six slit images achieved using the contracting-grid search method (4×4 grid with initial length of 7 mm and a contracting ratio of 0.75) is shown in FIG. 13. The total search time is 4.50 s to estimate 3×10⁵ events. If using the result of exhaustive search as ground truth, CG search has a standard deviation of 1.3 mm due to occasional large error by some events.

### 3.2.2. k-d Tree Search Method

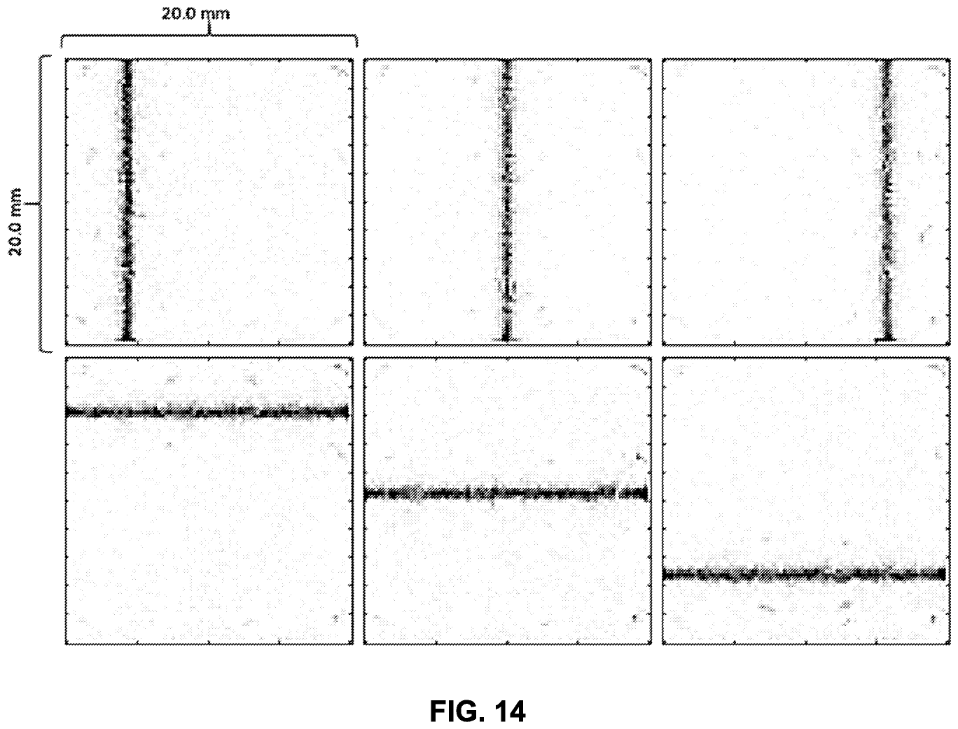

The k-d tree was constructed with the MDRFs achieved from the experiment calibration dataset. In this case, each entry in the k-d tree has 16 dimensions (since there are 16 SiPMs), and there are a total of 81×81 MDRF samples (corresponding to 6,561 leaves of the tree). The same six slit images (same data) of FIG. 13 estimated using the k-d tree search method are shown in FIG. 14. The total search time is 1.8 s to estimate 3×10⁵ events. Again, if using the result of exhaustive search as ground truth, k-d tree search has a standard deviation of 0.0 mm, which indicates an exact same result is achieved compared with exhaustive search method. This is not unexpected since the k-d tree is actually another kind of exhaustive search, but with branch pruning to increase the searching speed.

Note that each event from these experiments must be normalized before searching for its nearest neighbor in the k-d tree, since the MDRFs used to construct the k-d tree are normalized, the normalization is to keep them consistent with each other. The experimental results agree well with the simulation regarding precision and spent time for both methods. And by comparing with FIG. 12 (ground truth), these experiments indicates that the k-d tree search method has the highest precision with reduced searching time.

### Example 4. Discussion and Conclusion

Both the simulation and experimental results show the fact that the k-d tree search method can provide the highest search precision (same, or comparable, with an exhaustive search) with improved search speed as compared to CG search methods. Compared with contracting-grid search method, the k-d tree search method is more robust when searching in MDRFs with complicated merit function map (such as likelihood map), as the search precision is equivalent to exhaustive search.

The k-d tree search method actually belongs to the look-up table category. But compared with other look-up table methods, k-d tree searching has the highest accuracy (as good as exhaustive search). The algorithm is also relatively simple to implement, and almost fits well with any situation (for example, complicated MDRFs can be searched).

The time complexity of k-d tree search is O(log₂(N)), where N is the total number of entries (leaves) in the MDRFs or k-d tree (number of leaves in k-d tree is equal to number of entries of MDRFs), compared with time complexity O(N) of exhaustive search method, the search speed is greatly increased. Comparison of the search speed of both k-d tree and contracting-grid search method is difficult, as there are many parameters to adjust for contracting grid search method (such as number of trial positions in a grid, grid length, contracting ratio . . . ), but the k-d tree has reduced search speed in the examples in this work. The space complexity of k-d tree search is a little bit more than a contracting-grid search method, since both the nodes and leaves of k-d tree should be stored. But due to the fact that the node has only two attributes (index and value), while the leaves have k attributes, the increase of memory usage is not that great.

Since the k-d tree search method actually has exactly the same accuracy as exhaustive search method, the nearest neighbor of a query event is guaranteed to be found in decently short time (average search time is O(log 2(N))), where N is the number of entries in the reference dataset. The algorithm is also relatively simple to implement, and is robust to adapt to different situations (such as searching a detector with complicated MDRFs or using a different merit function to search). Moreover, this concept can be further extended into applications in other fields, such as locating interaction events in high-energy physics experiments (alpha and beta particles, energetic nucleus fragment or even neutrons), whenever a critical requirement on achieving both “accuracy and speed at the same time” is needed.

Still, similar to many other reference-data-based search methods, the apparent weakness of k-d tree search is the need of acquiring calibration data to construct the k-d tree, which could be very time-consuming. And the construction of k-d tree requires little bit more memory to store the nodes of the k-d tree (since the nodes requires less memory than leaves, the increase of memory use is small), while the leaves store the whole reference dataset. In addition, due to the many branches in the code of k-d tree search (many “if” and “else” statements, which create branches in the code), applying it on GPU is more challenging. In addition, the possible need to transport raw data (all readings of the light sensors of each event instead of its interaction position and energy) from detector modules to local computers will pose more harsh technical challenges on the communication bandwidth and data storage capacity. Despite these challenges, the high accuracy and fast searching capability of k-d tree search method facilitate its broad applications in SPECT/PET and high energy physics detectors, where fast and reliable localization of interaction events is required.

A comparison between the exhaustive search, contracting-grid search and k-d tree search in terms of accuracy and efficiency for the examples in the disclosure is summarized in the table below:

| | | Exhaustive | CG | | k-d tree | |
|---|---|---|---|---|---|---|
| | | search | search | | search | |
| Simulation | Standard deviation | N/A | 1.9 | mm | 0.0 | mm |
| | from ground truth | | | | | |
| | Search time for | 4292 s | 26 | s | 14 | s |
| | 9.3 × 10⁵ events | | | | | |
| Experiment | Standard deviation | N/A | 1.3 | mm | 0.0 | mm |
| | from ground truth | | | | | |
| | Search time for | 162 s | 4.5 | s | 1.8 | s |
| | 3.0 × 10⁵ events | | | | | |

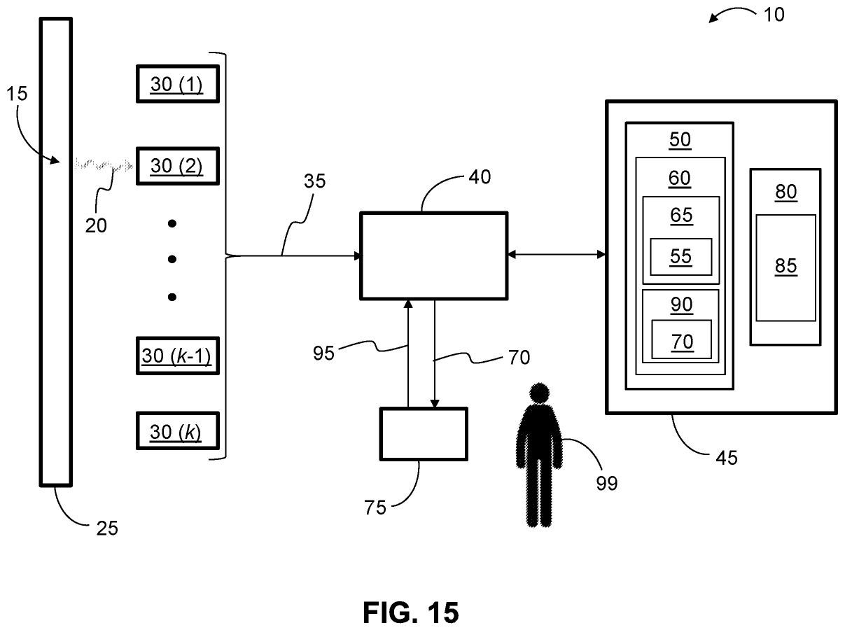

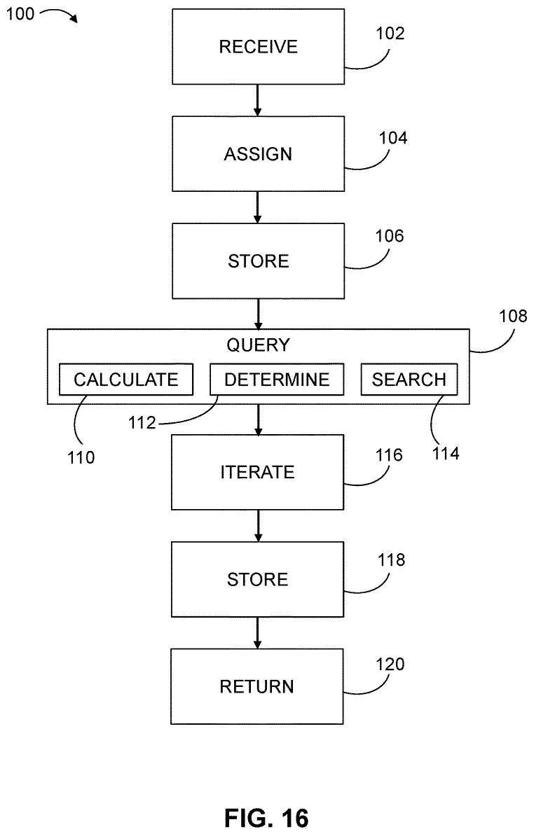

FIG. 15 shows a schematic diagram of a system 10 for estimating a position 15 of an interaction event, which generates scintillation photons 20, within a detector 25 in an embodiment of the invention. FIG. 16 shows a flow chart of a method 100 for estimating the position 15 of the interaction event within the detector 25 using signals 35 induced within the detector 25 induced by the scintillation photons 20 on one or more light sensors 30 in an embodiment of the invention.

System 10 includes k light sensors 30 (e.g., 30(1), 30(2), . . . , 30(k−1), and 30(k)) coupled in communication with one or more processors 40. System 10 includes one or more memory devices 45 coupled in communication with the processor(s) 40. In some embodiments, memory device(s) 45 include a non-transitory computer-readable medium 80 storing processor-executable program instructions as software 85. In embodiments in which one or more steps of method 100 are performed and/or implemented, at least in part, using software 85, processor(s) 40 executing the program instructions cause the processor(s) 40 to perform, implement, and/or otherwise facilitate one or more of the method 100 steps as disclosed herein. In the example shown, system 10 includes one or more input and/or output (I/O) device(s) 75 coupled in communication with processor(s) 40. Non-limiting examples of I/O devices 75 include displays, keyboards, mice, wired and/or wireless transceivers, and touchpads.

Method 100 includes receiving 102, from the k light sensor(s) 30 and by the processor(s) 40, a dataset 50 including o records 55 having values corresponding to the signals 35 induced by the scintillation photons 20. Method 100 includes assigning 104, by the processor(s) 40, a partition criteria to a k-dimensional (k-d) tree data structure 60 including n nodes 65 distributed over a plurality of levels (Lₘ). The n nodes 65 include: a root node assigned to first level (L₁) of the plurality of levels, and a plurality of intermediate nodes assigned to at least a second (L₂) and a third (L₃) level of the plurality of m levels (e.g., L₂, . . . Lₘ₋₁, Lₘ). The k-d tree data structure 60 includes a plurality of leafs 90 distributed in the k-d tree data structure 60 after a final level (Lₘ) of the plurality of levels. The root node, the plurality of intermediate nodes, and the plurality of leafs 90 are linked by branches. Method 100 includes storing 106, by the processor(s) 40 and in the memory device(s) 45, the o records 55 of the received 102 dataset 50 in the n nodes 65 of the k-d data structure 60. As used above, k is the number of light sensors, o is the number of records, m is the number of levels, and n is the number of nodes

Method 100 includes querying 108, by the processor(s) 40, the k-d tree data structure 60 for the position 15 of the interaction event. The querying 108 step includes: (a) calculating 110 a compound value from a query vector 95 (e.g., input using I/O device 75); (b) determining 112, by the processor(s) 40, a root node-to-second level intermediate node branch of the k-d tree data structure 60 to search based on a searching strategy; and (c) searching 114, by the processor(s) 40, the determined 112 root node-to-second level intermediate node branch of the k-d tree data structure 60. For the intermediate nodes of at least the third level of the k-d data structure 60, method 100 includes iterating 116, by the processor(s) 40, through the above-described calculating 110, determining 112, and searching 114 steps.

Method 100 includes storing 118, by the processor(s) 40 and in the memory device(s) 45, one or more resultant values 70 of the querying 108 step in at least one of the plurality of leafs 90. Method 100 includes returning 120, by the processor(s) 40, the resultant value(s) 70 stored 118 in the leaf(s) 90 as a result for the position 15 of the interaction event. In the example of FIG. 15, the one or more resultant values 70 are returned 120 by processor(s) 40 for viewing on a display device of the I/O device(s) 75 by a user 99 of system 10.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a size range, a number range, a pore size range, a porosity range, a thickness range, LOD range, a temperature range, a time range, a flow-rate range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicants invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A method for estimating a position of an interaction event generating scintillation photons within a detector using signals induced by the scintillation photons on k light sensors, where k is the number of light sensors, the method comprising:
receiving, from the k light sensors and by a processor communicatively coupled to the k light sensors, a dataset comprising o records having values corresponding to the signals induced by the scintillation photons, where o is the number of records;
assigning, by the processor, a partition criteria to a k-dimensional (k-d) tree data structure including n nodes distributed over a plurality of levels, where n is the number of nodes, wherein the n nodes, include: a root node assigned to a first level of the plurality of levels, and a plurality of intermediate nodes assigned to at least a second and a third level of the plurality of levels, wherein the k-d tree data structure further includes a plurality of leafs distributed in the k-d tree data structure after a final level of the plurality of levels, and wherein the root node, the plurality of intermediate nodes, and the plurality of leafs are linked by branches;
storing, by the processor and in a memory device communicatively coupled to the processor, the o records of the received dataset in the n nodes of the k-d tree data structure;
querying, by the processor, the k-d tree data structure for the position of the interaction event, wherein the querying step comprises: (a) calculating a compound value from a query vector; (b) determining, by the processor, a root node-to-second level intermediate node branch of the k-d tree data structure to search based on a searching strategy; (c) searching, by the processor, the root node-to-second level intermediate node branch of the k-d tree data structure selected by the searching strategy;
for intermediate nodes of at least the third level of the k-d data structure, iterating, by the processor, through steps (a), (b), and (c);
storing, by the processor and in the memory device, one or more resultant values of the querying step in one of the plurality of leafs; and
returning, by the processor, the one or more resultant values stored in the one of the plurality of leafs as a result for the position of the interaction event.

2. The method of claim 1, wherein the received dataset includes an array of values corresponding to the signals induced by the scintillation photons, and wherein the method further comprises constructing, by the processor, the k-d tree data structure from the array of values as a one-dimensional tree.

3. The method of claim 1, wherein the received dataset includes a plurality of vectors, each of the plurality of vectors including two or more values corresponding to the signals induced by the scintillation photons, and wherein the method further comprises constructing, by the processor, the k-d tree data structure from the plurality of vectors as a k-d tree having at least two-dimensions.

4. The method of any one of the preceding claims, further comprising constructing, by the processor, the k-d tree data structure as a binary search tree structure.

5. The method of any one of the preceding claims, further comprising:
determining, by the processor and based on the received dataset, mean detector response functions (MDRFs), the MDRFs defined as the mean signal responses of the k light sensors as functions of known interaction event positions; and
constructing, by the processor, the k-d tree data structure from the MDRFs.

6. The method of any one of the preceding claims, wherein the assigning step includes applying, by the processor, a rule for each of the n nodes, and wherein the querying step includes:
comparing, by the processor, the compound value calculated from the query vector for the querying step with one or more boundary values stored in the root node; and
selecting, by the processor and based on a result of the comparing step and the applied rule, one of a plurality of root node-to-second level intermediate node branches to search for the searching step,
wherein the iterating step includes applying, by the processor, the same rule for the intermediate nodes of the at least the third level of the k-d data structure.

7. The method of any one of the preceding claims, wherein the one or more boundary values include at least two boundary values, and wherein comparing the compound value calculated from the query vector for the querying step with one or more boundary values stored in the root node comprises determining that the compound value falls in an interval bounded by the at least two boundary values, or the compound value is less than the at least two boundary values, or the compound value is greater than the at least two boundary values.

8. The method of any one of the preceding claims, wherein selecting the one of a plurality of root node-to-second level intermediate node branches to search for the searching step comprises determining the one of a plurality of root node-to-second level intermediate node branches that satisfies the searching strategy.

9. The method of any one of the preceding claims, further comprising computing, by the processor: a distance, or a merit, between the one of the plurality of leafs storing the one or more resultant values of the querying step and the query for the querying step.

10. The method of any one of the preceding claims, wherein the interaction event is a gamma-ray, X-ray, neutron, proton, or other high energy particle interaction within the detector.

11. The method of any one of the preceding claims, wherein the interaction event is a gamma-ray interaction.

12. The method of any one of the preceding claims, wherein the receiving step includes receiving, by the processor, the dataset from k light sensors of a single-photon emission computed tomography (SPECT) scanner.

13. The method of any one of the preceding claims, wherein the receiving step includes receiving, by the processor, the dataset from k light sensors of a positron emission tomography (PET) scanner.

14. The method of any one of the preceding claims, wherein the compound value is a likelihood value or likelihood reduction value.

15. A system for estimating a position of an interaction event generating scintillation photons within a detector, the system comprising:
light sensors for generating signals induced by scintillation photons incident thereupon;
a memory device;
a processor communicatively coupled to the light sensors and to the memory device, wherein the processor is programmed to:
receive, from the light sensors, a dataset including values corresponding to the signals induced by the scintillation photons;
assign a partition criteria to a k-d tree data structure having nodes and leafs, wherein the nodes include a root node, intermediate nodes, and penultimate nodes, wherein the root node, the intermediate nodes, the penultimate nodes, and the leafs are linked by branches;
store, in the memory, the received dataset to the k-d tree data structure;
querying, by the processor, the k-d tree for the position of the interaction event position, wherein the querying step comprises: (a) calculate a compound value from a query vector; (b) determine a branch of the k-d tree to search based on a searching strategy; (c) search the branch of the k-d tree selected by the searching strategy;
for the intermediate nodes and the penultimate nodes, iterate through processor operations (a), (b), and (c); and
return the value stored in the leaf as a query result for the position of the interaction event position.

16. The system of claim 15, wherein the received dataset includes an array of values corresponding to the signals induced by the scintillation photons, and wherein the processor is further programmed to construct the k-d tree data structure from the array of values as a one-dimensional k-d tree.

17. The system of claim 15, wherein the received dataset includes a plurality of vectors, each of the plurality of vectors including two or more values corresponding to the signals induced by the scintillation photons, and wherein the processor is further programmed to construct the k-d tree data structure from the plurality of vectors as a k-d tree having at least two-dimensions.

18. The system of any one of claims 15-17, wherein the processor is further programmed to construct the k-d tree data structure as a binary search tree structure.

19. The system of any one of claims 15-18, wherein the processor is further programmed to:
determine, based on the received dataset, mean detector response functions (MDRFs), the MDRFs defined as the mean signal responses of the light sensors as functions of known interaction positions; and
construct the k-d tree data structure from the MDRFs.

20. The system of any one of claims 15-19, wherein, for the assigning processor operation, the processor is further programmed to apply a rule for each of the nodes, and wherein, for the querying processor operation, the processor is further programmed to:
compare a value calculated from the query with one or more values stored in the root node; and
select, based on a result of the comparing processor operation and the applied rule, one of two or more branches to search for the searching processor operation,
wherein, for the iterating processor operation, the processor is further programmed to apply the same rule for the intermediate nodes and the penultimate nodes.

21. The system of any one of claims 15-20, wherein the processor is further programmed to compute a distance (or merit) between the leaf storing the value of the query result and the query for the querying processor operation.

22. The system of any one of claims 15-21, wherein the interaction event is a gamma-ray, X-ray, neutron, proton, or other high energy particle interaction within the detector.

23. The system of any one of claims 15-22, wherein the interaction event is a gamma-interaction.

24. The system of any one of claims 15-23, further comprising a medical imaging system including the light sensors, wherein, for the receiving processor operation, the processor is further programmed to receive the dataset from light sensors of medical imaging system.

25. The system of claim 24, wherein the medical imaging system is a single-photon emission computed tomography (SPECT) scanner including the light sensors, and wherein, for the receiving processor operation, the processor is further programmed to receive the dataset from light sensors of SPECT scanner.

26. The system of claim 24, wherein the medical imaging system is a positron emission tomography (PET) scanner including the light sensors, wherein, for the receiving processor operation, the processor is further programmed to receive the dataset from light sensors of PET scanner.

27. The system of any one of claims 15-26, wherein the compound value is a likelihood value or likelihood reduction value.

28. A non-transient computer readable medium comprising processor-executable instructions stored therein for estimating a position of an interaction event generating scintillation photons within a detector, which, when executed by one or more processors communicatively coupled to: one or more memory devices, and light sensors for generating signals induced by scintillation photons incident thereupon, cause the one or more processors to:
receive, from the light sensors, a dataset including values corresponding to the signals induced by the scintillation photons;
assign a partition criteria to a k-d tree data structure having nodes and leafs, wherein the nodes include a root node, intermediate nodes, and penultimate nodes, wherein the root node, the intermediate nodes, the penultimate nodes, and the leafs are linked by branches;
store, in the memory, the received dataset to the k-d tree data structure;
query the k-d tree for the position of the interaction event position, wherein, for querying the k-d tree, the processor-executable program instructs the one or more processors to: (a) calculate a compound value from a query vector; (b) determine a branch of the k-d tree to search based on a searching strategy; (c) search the branch of the k-d tree selected by the searching strategy;
for the intermediate nodes and the penultimate nodes, iterate through the processor operations (a), (b), and (c); and
return the value stored in the leaf as a query result for the position of the interaction event position.

29. The non-transient computer readable medium of claim 28, wherein the compound value is a likelihood value or likelihood reduction value.

30. The non-transient computer readable medium of claims 28-29, wherein the interaction event is a gamma-ray, X-ray, neutron, proton, or other high energy particle interaction within the detector.

31. The non-transient computer readable medium of claims 28-30, wherein the interaction event is a gamma-ray interaction.
